(19) United States
(12) Patent Application Publication
(10) Pub. No.: US 2020/0254011 A1
(43) Pub. Date: Aug. 13, 2020

(54) NANOCOMPOSITE FORMULATION FOR USE IN HEMOSTASIS

(71) Applicant: INDIAN INSTITUTE OF TECHNOLOGY, BOMBAY, Mumbai (IN)

(72) Inventors: Rinti Banerjee, Mumbai (IN); Chandrashekhar Prasad, Mumbai (IN); Kiran Jadhav, Mumbai (IN)

(21) Appl. No.: 16/751,476

(22) Filed: Jan. 24, 2020

(30) Foreign Application Priority Data
Feb. 8, 2019 (IN) 201921005029

(51) Int. Cl. A61K 33/06; A61K 31/715; A61K 9/06; A61K 9/14; A61K 9/20

(52) U.S. Cl. CPC A61K 33/06; A61K 31/715; A61K 9/20; A61K 9/14; A61K 9/06

(57) ABSTRACT

The present invention relates to a nanocomposite formulation for use in hemostasis comprising at least one calcium-silicate and at least one polysaccharide. The invention also relates to solvent-free process for preparation of the nanocomposite formulation.

### CROSS-REFERENCE TO RELATED PATENT APPLICATION

This application claims priority to and the benefit of Indian Patent Application No. 201921005029, filed Feb. 8, 2019, which is incorporated herein in its entirety by reference.

### FIELD OF THE INVENTION

This invention refers to a formulation for point of care ready to use haemostats which hasten the clotting of blood and prevent the chances of advancement in the severity of the patient's condition due to haemorrhage. The invention specifically refers to nanocomposite formulations consist of combination of calcium-silicate nanoparticles in a biopolymeric matrix which have a wide range of application as anti-haemorrhagic agents in both civilian accidents and haemorrhage associated with army personnel in battle field.

### BACKGROUND OF THE INVENTION

Uncontrollable haemorrhage is the leading cause of death in battle-field which predominantly affects the limbs and becomes more challenging in a remote battle field where the immediate medical care is difficult to achieve. There is an urgent need to stabilise the soldier temporarily at the field, to prevent life threatening blood loss, infections and pain which can enable the soldier to complete first aid task prior to being transfened for hospital. To circumvent such problems, various polymers have been used and are under examination to be used as hemostatic agent for the fast clotting of blood in accidental haemorrhage. Globally, Celox® Gauze, Chito-SAM® and QuickClot® are marketed products which are being used to stop lethal bleeding. Celox® Gauze and Chito-SAM® are Z folded chitosan impregnated bandages which are applied on wound site with pressure. These products are in both Gauze and granular form but granular forms are not easy to handle and apply in terms of its deposition over specific area. Gauze forms are better in that respect but require an additional person to push the Gauze deeper in the wound. Animal studies and case reports revealed that QuickClot® is poorly biodegradable and induce thermal injuries/burning sensation at the site of application. Even if it has proven to be quite promising hemostat in venous haemorrhage and mixed arterial-venous bleeding it failed to show any benefit on arterial injury model. QuickClot ACS® (QC, zeolite based hemostat) is improved version of QuickClot® which is easy to handle and does not induce any thermal sensation but is not arterial hemostatic. None of these dressings are antibacterial or pain relieving and are available as bulky gauze or granules which are difficult to self administer. All existing hemostats need improvement in terms of their ability of preventing arterial haemorrhage.

CN1330389C claims development of hemostatic gel comprising polylactic acid (PLA), protein and polysaccharide hemostatic drug and polyvinyl pyrrolidone. The amount of constituent was varying, viz. for pyrrolidone it was extending from 10% to 93%, for protein and polysaccharide it was varying from 5% to 65% and for PLA it was varying from 0% to 30%. All the components were mixed together to form the hemostatic gel. The inventors claim that application of gel over injury leads to the generation of non-adhesive thin film which stops haemorrhagic loss of blood. The patent teaches a composition of synthetic polymers and proteins as a hemostat.

US20070237811A1 discloses use of chitosan as a biopolymeric hydrogel or foam that can be layered onto appropriate backing to be used as wound dressing. The claims also propose the addition of anti-inflammatory, anti-viral, anti-fungal, collagen, maltodextrin and anti-bacterial agents to increase the effectiveness as wound dressing and hemostatic agent. 29 gm of chitosan with 200,000 centipoise (cps) viscosity was dissolved in 550 ml of 8% acetic acid prepared in deionised water. Sodium bicarbonate in the amount of 19.2 gm was added to the chitosan-acetic acid mixture under continuous stirring to generate foam within chitosan gel. Chitosan foamed gel can be sterilized and filled inside squeezable tube and dispensed out over injury to stop bleeding. Although the inventors have given clear methodology to develop gel and explained the vital role of constituent in making embodiments a hemostatic wound dressing. But, neither in vitro nor in vivo validation of the embodiments had been done to prove the efficacy of the formulation. The patent teaches a composition of chitosan requiring acetic acid for its preparation.

WO2007074326A1 protects hemostatic powder consists of chitosan salt and at least one inert material and medical surfactant. Proportion of chitosan in hemostatic powder was 20% by weight, inert material 30% by weight where as medical surfactant constitute 0.01% by weight. In order to prepare chitosan salt, chitosan was mixed with acid (e.g. succinic acid) in a solvent comprised 80:20 (v/v) ratio of ethanol: water. Medical surfactant viz. lauric acid or oleic acid was also added to the mixture. Blend was mixed properly in dough style mixture for 15 mins to change it into slurry. Slurry, thus formed, was dried at 60° C. to evaporate the solvent. Solid chunk obtained was passed through grinding mill to make uniform fine powder. This chitosan salt powder was then mixed with dry inert powder, viz. cellulose, fumed silica, alginate, sand, clay, microcrystalline cellulose, to produce final hemostat. The patent teaches a combination of chitosan and surfactants and requires ethanol during the formation.

CN1044745856A invention refers to a biological hemostatic gel made up of peanut coat and chitosan powder. It claims 0.8-1.3% of soluble chitosan, 1-2.6% of peanut coat powder of red peanuts, 0.3-0.8% of panax pseudoginseg powder, 0.2-0.3% of chlorhexidine acetate, 0.8-1.3% of carbomer and 93.7-96.9% of deionized water. In order to prepare hemostatic gel, deionized water was heated till 55-60° C. and then 0.2-0.3% of chlorhexidine acetate was added to increase the acidity of the solution which increased the rate of 0.8-1.3% chitosan dissolution. After stirring for 15 mins, 1-2.6% of peanut powder, 0.3-0.8% of panax pseudoginseg powder and 0.8-1.3% of carbomer was added and further stirred for 30 mins to dissolve them. The inventors claim that the invention can be directly applied over wound/injuries to stop the bleeding. Though the inventors have claimed that it can increase the rapid haemostasis but neither in vitro nor in vivo study was reported to back the idea of developing this particular hemostat. The patent teaches a multicomponent formulation of chitosan, peanuts and ginseng powders and requires acidic conditions for dissolution.

### SUMMARY OF THE INVENTION

In one aspect, the present invention provides a nanocomposite formulation for use in hemostasis comprising at least one calcium-silicate nanoparticles and at least one polysaccharide.

In another aspect, the present invention provides a solvent free method of preparation of a nanocomposite formulation for use in hemostasis comprising at least one calcium-silicate nanoparticles and at least one polysaccharide.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1A shows a scanning electron microscope image of calcium-silicate nanoparticles in accordance with an embodiment of the invention. FIG. 1B shows energy dispersive x-ray spectrum showing elemental composition of calcium-silicate nanoparticles in accordance with an embodiment of the invention.

FIG. 2 shows a schematic representation of the blood clotting in presence of different forms of hemostats like smart fabric, pellets, flakes, powder and gel in accordance with an embodiment of the invention.

FIG. 3 shows a scanning electron microscope image of hemostatic flakes consisting of 4% calcium-silicate nanoparticles and 1% gellan in accordance with an embodiment of the invention.

FIG. 4A shows a scanning electron microscopic image of gel, 4% calcium-silicate nanoparticles +1% gellan, in accordance with an embodiment of the invention. FIG. 4B shows a scanning electron microscopic image of gel, 5% calcium-silicate nanoparticles +1% gellan, in accordance with an embodiment of the invention.

FIG. 5A shows clotting time of blood in presence of gel (4% calcium-silicate nanoparticles +1% gellan) and comparison with marketed formulation celox®, in accordance with an embodiment of the invention. FIG. 5B shows clotting time of blood in presence of gel (5% calcium-silicate nanoparticles +1% gellan) and comparison with marketed formulation celox®, in accordance with an embodiment of the invention.

FIG. 6A shows smart fabric, in accordance with an embodiment of the invention. FIG. 6B shows 5 inch×2 inch of smart fabric absorbed 10 ml of blood within 30 sec and clotted the blood in 3±0.3 mins, in accordance with an embodiment of the invention. FIG. 6C shows 5 inch×2 inch of marketed cotton bandage could not absorb 10 ml blood, in accordance with an embodiment of the invention.

FIG. 7A shows clotting of 10 ml goat blood in presence of 6 pellets, in accordance with an embodiment of the invention. FIG. 7B shows residence time of pellets inside blood; a pellet dipped in 25 ml of goat blood was still intact after 2.5 hours incubation in blood, in accordance with an embodiment of the invention. FIG. 7C shows 250 mg of celox® neither absorbed nor clotted the 10 ml of citrated goat blood, in accordance with an embodiment of the invention.

FIG. 8 shows Prothrombin time (PT) of gel, powder, pellets, flakes and celox® in human plasma in accordance with an embodiment of the invention.

FIG. 9 shows activated partial thromboplastin time (APTT) of gel, powder, pellets, flakes and celox® in human plasma in accordance with an embodiment of the invention

FIG. 10A shows hemostats in the form of pellets, in accordance with an embodiment of the invention. FIG. 10B shows hemostats in the form of flakes, in accordance with an embodiment of the invention. FIG. 10C shows hemostats in the form of powder, in accordance with an embodiment of the invention. FIG. 10D shows hemostats in the form of gel, in accordance with an embodiment of the invention.

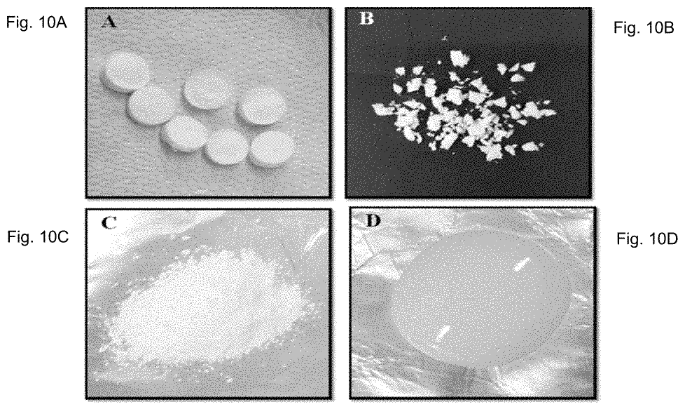

FIG. 11 shows In vivo blood clotting study in Sprague-dawley rat using tail amputation model. Graph shows time required for hemostasis, left panel. Graph shows amount of blood released till clot formation occurred, right panel in accordance with an embodiment of the invention.

### DETAILED DESCRIPTION OF THE INVENTION

Definition: “Nanocomposites” are combination of phosphorus-calcium-silicate nanoparticles in a biopolymeric matrix.

The present invention is directed towards a formulation for use in hemostasis comprising at least one calcium-silicate nanoparticles and at least one polysaccharide especially designed for deep wound and is meant to push inside wound/injury to stop the blood loss. These hemostats are self administrable in nature and clot the blood instantly by transforming into a highly cross-linked semi-solid mass upon mixing with blood.

In one aspect, the invention provides a nanocomposite formulation for use in hemostasis comprising at least one calcium-silicate nanoparticle and at least one polysaccharide.

In an embodiment, the calcium-silicate nanoparticle is in the size range of 40-150 nm.

In another embodiment, the polysaccharide is selected from gellan, Carboxymehtyl cellulose, Xanthan, Alginate and carbopol.

The calcium-silicate nanoparticles may be present in the range of 4-7% w/v.

The polysaccharide may be present in the range of 0.1-2% w/v.

The formulation may be in the form of gel, dry flakes, powder, coated on patch/fabric or developed as hemostatic uniform/garments through stitching.

The present invention is further directed towards a solvent free process for the preparation of a formulation comprising blending of at least one calcium-silicate nanoparticles and at least one polysaccharide in a specific ratio at a predetermined temperature.

In an embodiment the blending may by mortar and pestle, sonication or homogenization.

In an embodiment, the calcium-silicate nanoparticle is in the size range of 40-150 nm.

In another embodiment, the polysaccharide is selected from gellan, Carboxymehtyl cellulose, Xanthan, Alginate and carbopol.

The ratio of calcium-silicate nanoparticles and polysaccharide is in the range of 4-7 : 0.1-2 w/v %, particularly in the ratio of 4:1 or 5:1 w/v %.

The blending is carried out at a temperature of 25-90° C.

### EXAMPLES

The following experimental examples are illustrative of the invention but not limitative of the scope thereof:

### Example 1

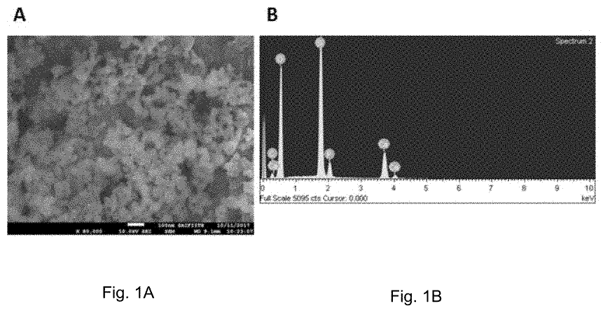

Solution 1 was prepared by dissolving 2.36 gm of calcium nitrate and 5.47 ml of tetraethyl orthosilicate in ethanol:water (1:1, v/v) solution. Then the pH was adjusted to 1-2 with the help of 1-2 ml of nitric acid. Solution 2 was prepared by dissolving 0.33 gm of ammonium dibasic phosphate in 1200 ml of MilliQ and pH was adjusted to 10-11 with the help of 5-6 ml of ammonia water. Solution 2 was kept for stirring and solution 1 was added to it slowly to obtain a milky solution. This milky solution was kept under stirring for 12 hours at room temperature and thereafter it was centrifuged at 10000×g for 15 min at 10° C. Pellet was resuspended in MilliQ and the centrifugation was repeated 2-3 times to obtain the pure calcium-silicate nanoparticles. This pure calcium-silicate nanoparticles was dried through lyophilization/freeze drying (freezing at −196° C. for 20 mins and drying at −40° C.) and further calcinated at 500° C. in muffle furnace for 3 hours to obtain the crystalline calcium-silicate nanoparticles (size 60-80 nm) comprised weight percent of 4.54, 20.26, 30.59 and 39.37% for P, Ca, Si and O respectively (FIGS. 1A and 1B). These calcium-silicate nanoparticles were used to make the different type of hemostats.

### Example 2

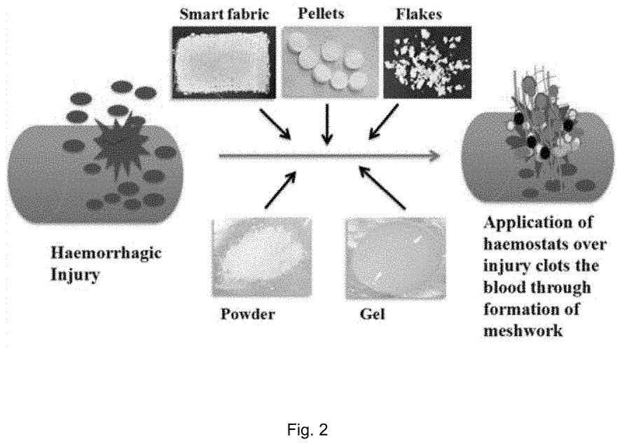

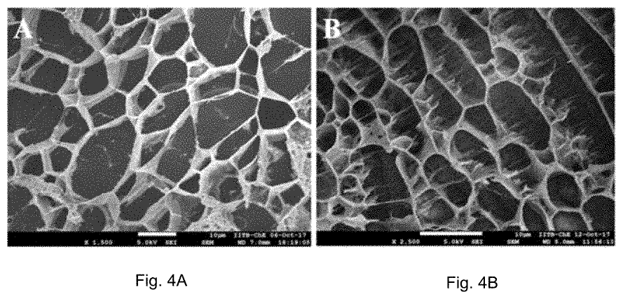

Developed calcium-silicate nanoparticles (5%, w/v) were blended with gellan (1%, w/v; purity ≥80%) at temperature 25° C. in 5 ml MilliQ with the help of mortar-pestle to form hemostatic gel (FIGS. 2 and 4B). For blending of constituents sonication (at power 80 W, on time 3 sec and off time 1 sec for 4 min duration) was also tried.

### Example 3

Developed calcium-silicate nanoparticles (4%, w/v) were blended with gellan (1%, w/v) at temperature 25° C. in 5 ml MilliQ with the help of mortar-pestle to form hemostatic gel (FIGS. 2 and 4A). For blending of constituents sonication (at power 80 W, on time 3 sec and off time 1 sec for 4 min duration) was also tried.

### Example 4

Developed calcium-silicate nanoparticles and gellan were blended together in ratio of 4:1 (wt/wt) at temperature 25° C. to obtain hemostatic powder (FIG. 2).

### Example 5

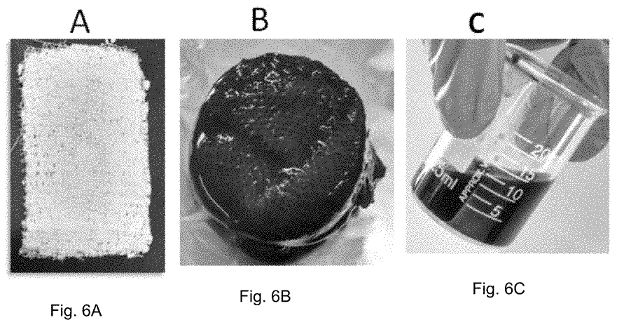

Developed calcium-silicate nanoparticles (4%, w/v) were blended with gellan (1%, w/v) at temperature 25° C. in 5 ml MilliQ using mortar-pestle. For blending of constituents sonication (at power 80 W, on time 3 sec and off time 1 sec for 4 min duration) was also tried. Gel was coated over three layers of cotton bandage. After that it was covered with parafilm and allowed to air dry for 2 days to develop smart fabric (FIG. 6A).

### Example 6

Developed calcium-silicate nanoparticles (4%, w/v) were blended with gellan (1%, w/v) at temperature 25° C. in 5 ml MilliQ using mortar-pestle. For blending of constituents sonication (at power 80 W, on time 3 sec and off time 1 sec for 4 min duration) can also be used. 1 ml of hemostatic gel was transferred to each well of 24 well cell culture plates. It was then lyophilized/freeze drying (freezing at −196° C. for 20 mins and drying at −40° C.) for 24 hours to generate tablet shape hemostatic pellet (FIG. 2).

### Example 7

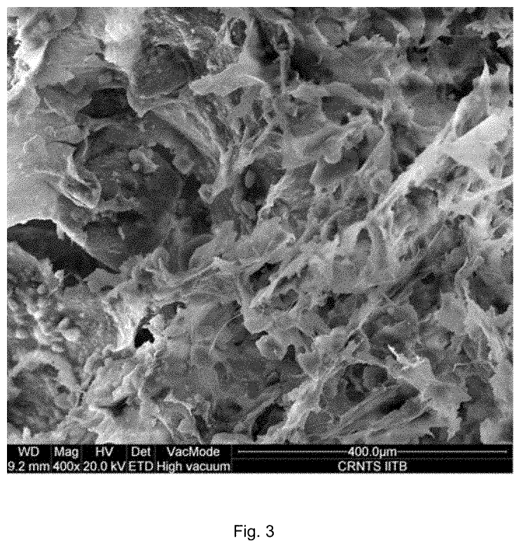

Developed calcium-silicate nanoparticles (4%, w/v) were blended with gellan (1%, w/v) at temperature 25° C. in 5 ml MilliQ using mortar-pestle. For blending of constituents sonication (at power 80 W, on time 3 sec and off time 1 sec for 4 min duration) can also be used. 1 ml of hemostatic gel was transferred to each well of 24 well cell culture plates. It was then lyophilized/freeze drying (freezing at −196° C. for 20 mins and drying at −40° C.) for 24 hours to generate tablet shape hemostatic pellet. These pellets were broken down into small flakes (FIG. 2). Scanning electron microscope image of flakes is shown in FIG. 3.

### Example 8

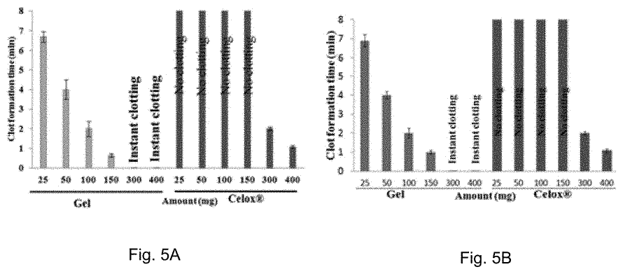

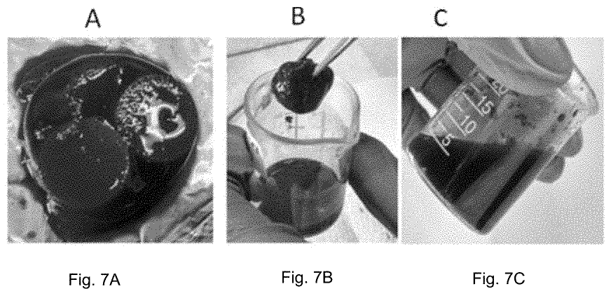

The hemostatic activity of gel (5% calcium-silicate nanoparticle and 1% gellan) was evaluated using 3 ml of citrated goat blood and clotting time was recorded by tilting the tube on regular intervals. The clotting time was found to be 6.9±0.32, 4±0.20, 2±0.25, 1±0.10 min for 25, 50, 100, 150 mg of gel respectively (FIG. 5B). Moreover, 300 and 400 mg of gel clotted the blood instantly just after addition. Contrastingly, 25, 50, 100, 150 mg of celox® could not clot the same volume of blood. Although, 300 and 400 mg of celox® did clot the same volume of blood in 2±0.09 and 1.08±0.07 min respectively. So, it is now evident that present invention was clotting the blood faster than the marketed formulation and required in comparatively less amount for clotting the same volume of blood. The hemostatic activity of gel (4% calcium-silicate nanoparticle and 1% gellan) was also evaluated using 3 ml of citrated goat blood and clotting time was recorded by tilting the tube on regular intervals. The clotting time was found to be 6.7±0.26, 4±0.5, 2±0.4 and 0.66±0.08 min for 25, 50, 100 and 150 mg of gel (FIG. 5A). Clotting time of blood in presence of celox® was also evaluated and it was found that the 25, 50, 100 and 150 mg could not clot the blood but 300 and 400 mg of celox® clotted the blood in 2±0.09 min and 1.1±0.07 min respectively (FIG. 5A). Moreover, the hemostatic activity of smart fabric with dimension 5 inch×2 inch was determined in citrated goat blood and it was found that it absorbed the 10 ml of blood within 30 sec and clotted the whole blood in 3±0.3 mins (FIG. 6B). But, the same dimension of marketed cotton gauze neither absorbed nor clotted the 10 ml blood (FIG. 6C). The pellets were also evaluated for hemostatic activity using citrated goat blood. It was found that 6 pellets were able to clot the 10 ml blood in 2±0.2 min (FIG. 7A). These pellets were also evaluated for its residence time by dipping pellet in 25 ml of blood. It was observed till 2.5 hours and it was found that pellet was physically intact (FIG. 7B). Non-dissolution of pellet inside blood even after such a long duration makes it quite useful as hemostatic agent for deep wound.

### Example 9

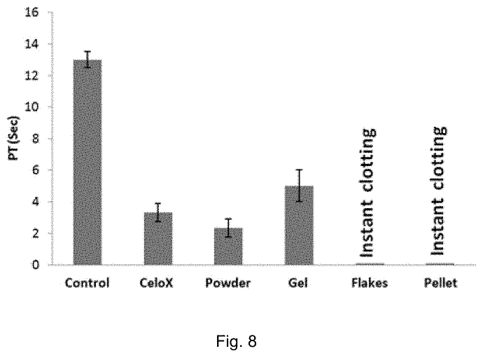

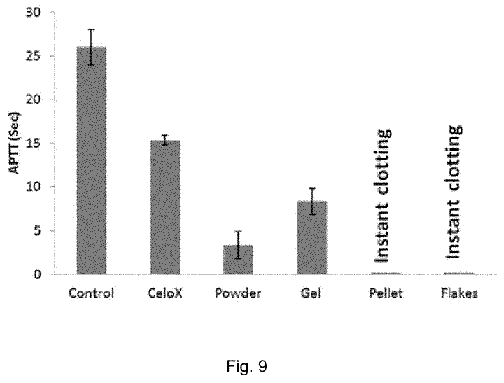

The gel (4% calcium-silicate nanoparticle and 1% gellan) was also evaluated for hemostatic parameters like prothrombin time (PT) and activated partial thromboplastin time (APTT) in human plasma and it was found to be 5±1 and 8.3±1.5 sec respectively. PT of gel was found to be higher than celox® but lower than control. However, APTT time of gel was lower than both control and celox®. Hemostatic powder was tested for its hemostatic activity with human plasma. 15 mg of this powder was used to determine the PT and APTT with human plasma. The PT and APTT of the powder was found to be 2.3±0.6 and 3.3±1.5 sec respectively whereas for celox® it was found to be 3.3±0.6 and 15.3 ±0.6 sec respectively. Experiment showed that both PT and APTT of plasma mixed with powder was less than both, control and celox®. The developed pellets were tested for its hemostatic activity with human plasma. 15 mg of pellets were also tested for PT and APTT with human plasma and found that pellets clotted the entire plasma instantly whereas PT and APTT of celox® was found to be 3.3±0.6 and 15.3±0.6 sec respectively. Developed flakes were also tested for PT and APTT in human plasma and it was found that 15 mg flakes clotted the entire plasma instantly (FIGS. 8 and 9). Here control refers to PT and APTT of human plasma without addition of any hemostat.

### Example 10

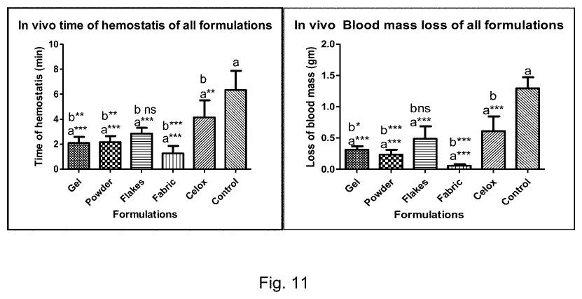

The in vivo blood clotting ability of all the hemostats was evaluated through tail amputation model (n=6, 6-8 weeks old) in albino wistar rat. Tail of each rat was amputated and thereafter formulation was applied. Amount of blood released as well as the time required to stop the blood loss was determined. The time required to clot the blood and the released blood mass is shown in FIG. 11. As we can see in FIG. 11 that the time required for hemostasis as well as the amount of blood released before clot formation occurred is lower in flakes, powder, gel, pellet and fabric than both celox® and control.

### Example 11

Fixed dose acute dermal toxicity of the hemostats was determined in GLP facility as per OECD guidelines in Sprague Dawley rats. Hemostatic gel was tested for the dose of 50-2000 mg/kg body weight and no clinical sign and mortalities were observed at any of the tested dose. Hence, it is concluded that the acute dermal median lethal dose (LD₅₀) of hemostatic gel in Sprague Dawley rats is>2000 mg/kg body weight and classified as “Category 5/Unclassified” (2000<ATE≤5000 mg/kg body weight) according to the Globally Harmonized System (GHS) of Classification.

The formulations of the present invention in the form of gel, powder, pellet, flakes and smart fabric have shown better hemostatic activity than brand names/marketed formulation celox®. Moreover, the dose required to clot the similar volume of blood was lower in our formulation as compared to marketed celox®. The developed smart fabric can be used for both superficial and deep wound due its quick clotting time and extensive blood absorption capacity. This fabric can be stitched over the sleeves of garments/uniform and the injured person can just snatch it off and apply over the injury. The gel, flakes, powder, smart fabric and pellets are hemostatic agents for superficial wound but smart fabric, gel and pellets are meant to be used for deep wound. Moreover, the longer residence time of pellet would enable the complete removal of pellet. Also, the in vivo hemostasis study showed that the developed formulation were quicker in clotting the blood than marketed celox®. The developed hemostats were non toxic for topical applications as we observed in acute dermal toxicity study. Hence, all the hemostats can be used as anti-haemorrhagic agent in both civilian accidents and haemorrhage associated with army personnel in battle field.

The foregoing description of the invention has been set merely to illustrate the invention and is not intended to be limiting. Since the modifications of the disclosed embodiments incorporating the spirit and substance of the invention may occur to the person skilled in the art, the invention should be construed to include everything within the scope of the disclosure.

What is claimed is:

1. A nanocomposite formulation for use in hemostasis comprising at least one calcium-silicate and at least one polysaccharide.

2. The nanocomposite formulation as claimed in claim 1, wherein the calcium-silicate is in the form of nanoparticle with the size range of 40-150 nm.

3. The nanocomposite formulation as claimed in claim 1, wherein the weight percent of phosphorus, calcium, silica and oxygen in calcium-silicate nanoparticles is in the range of 1-20%, 10-45%, 20-50 and 25-60% respectively.

4. The nanocomposite formulation as claimed in claim 1, wherein the polysaccharide is selected from gellan, Carboxymehtyl cellulose, Xanthan, Alginate and carbopol.

5. The nanocomposite formulation as claimed in claim 1, wherein the calcium-silicate nanoparticles is in the range of 4-7% w/v.

6. The nanocomposite formulation as claimed in claim 1, wherein the polysaccharide is in the range of 0.1-2% w/v.

7. The nanocomposite formulation as claimed in claim 1, wherein the formulation is in the form of gel, dry flakes, powder, coated on patch/fabric or developed as hemostatic uniform/garments through stitching.

8. A solvent free process for the preparation of a nanocomposite formulation comprising blending of at least one calcium-silicate nanoparticles and at least one polysaccharide in a specific ratio at a predetermined temperature.

9. The solvent free process as claimed in claim 8, wherein the blending is by mortar and pestle, sonication or homogenization.

10. The solvent free process as claimed in claim 8, wherein the calcium-silicate is in the form of nanoparticles with size range of 40-150 nm.

11. The solvent free process as claimed in claim 8, wherein the polysaccharide is selected from gellan, Carboxymehtyl cellulose, Xanthan, Alginate and carbopol.

12. The solvent free process as claimed in claim 8, wherein the ratio of calcium-silicate nanoparticles and polysaccharide is in the range of 4-7: 0.1-2 w/v %, particularly in the ratio of 4:1 or 5:1 w/v %.

13. The solvent free process as claimed in claim 8, wherein the process further comprises the step of drying.

14. The solvent free process as claimed in claim 13, wherein the drying is carried out by simple heating in furnace or heat drier at 100-700° C.

15. The solvent free process as claimed in claim 13, wherein the drying is in a vacuum furnace with heating at (100-700° C.).
